(19) United States
(12) Patent Application Publication
(10) Pub. No.: US 2017/0153439 A1
(43) Pub. Date: Jun. 1, 2017

(54) CONNECTION DEVICE AND IMAGING APPARATUS PROVIDED WITH CONNECTION DEVICE

(71) Applicant: TERUMO KABUSHIKI KAISHA, Tokyo (JP)

(72) Inventor: Toyokazu HORIIKE, Shizuoka (JP)

(73) Assignee: TERUMO KABUSHIKI KAISHA, Tokyo (JP)

(21) Appl. No.: 15/430,922

(22) Filed: Feb. 13, 2017

Related U.S. Application Data
(63) Continuation of application No. PCT/JP2015/072764, filed on Aug. 11, 2015.

(30) Foreign Application Priority Data
Aug. 14, 2014 (JP) 2014-165228

(51) Int. Cl. G02B 23/24; A61B 8/12; G02B 6/38; A61B 8/00; A61B 5/00; G02B 23/26

(52) U.S. Cl. CPC G02B 23/2453; A61B 5/0066; A61B 5/6852; A61B 5/742; G02B 23/26; G02B 6/3874; A61B 8/56; A61B 8/12; A61B 8/4483; A61B 5/6876

(57) ABSTRACT

A connection device is disclosed in which information transmission cables can be reliably connected to each other while jamming is prevented from occurring in the connection device, and an imaging apparatus provided with the connection device. The connection device can include an adapter fixing member internally having a guide member. Inside the guide member, there is provided a helical guide surface formed in one direction in order to perform an operation in which a projection portion of a connector fixing member is guided in a manner of gyrating around a central axis of a first optical fiber cable as a first information transmission cable and a second optical fiber cable as a second information transmission cable, when the connector fixing member is inserted.

### CROSS-REFERENCES TO RELATED APPLICATIONS

This application is a continuation of International Application No. PCT/JP2015/072764 filed on Aug. 11, 2015, which claims priority to Japanese Application No. 2014-165228 filed on Aug. 14, 2014, the entire contents of which are incorporated herein by reference.

### TECHNICAL FIELD

The present disclosure relates to a connection device for connecting information transmission cables to each other, and an imaging apparatus provided with the connection device.

### BACKGROUND

Recently, as the large-capacity communication advance, mainly in the telecommunications field, a connection device for optical fiber cables has been adopted in order to connect optical fiber cables to each other.

In the medical field, various types of medical instruments such as an optical coherence tomography apparatus (OCT) are used in order to perform medical diagnosis or treatment utilizing light. In these medical instruments, for example, light is utilized as a signal, and the optical fiber cables are used when transmitting an optical signal. Therefore, in the field of medical instruments, the connection device is inevitably used in order to connect the optical fiber cables to each other.

U.S. Pat. No. 8,322,932 discloses an optical fiber cable connection device disposed in an optical imaging apparatus. As illustrated in FIG. 12 of U.S. Pat. No. 8,322,932, the connection device includes a connector member 405 that is connected to a catheter side, and an adapter fixing member 603 inside a motor drive unit (MDU). The connector member 405 to be connected to the catheter connects end portions of optical fiber cables to each other by being inserted into the adapter fixing member 603. As illustrated in FIG. 9 of U.S. Pat. No. 8,322,932, the connector member 405 has a protruding portion 702 on an outer peripheral surface. In addition, as illustrated in FIG. 8, a guide tube 609 inside the adapter fixing member 603 has slope end surfaces 801A and 801B respectively in two directions bifurcated from the vertex, and a cut-off portion 802.

As illustrated in FIG. 12 of U.S. Pat. No. 8,322,932, when the connector fixing member 405 is inserted into the adapter fixing member 603 and the end portions of the optical fiber cables are connected to each other, the protruding portion 702 of the connector fixing member 405 is guided in a manner of gyrating along any one of the slope end surfaces 801A and 801B of the adapter fixing member 603. Accordingly, the connector fixing member 405 and the adapter fixing member 603 relatively rotate, and the protruding portion 702 of the connector fixing member 405 is fitted into the cut-off portion 802 of the adapter fixing member 603.

In this manner, when the connector fixing member 405 and the adapter fixing member 603 are connected to each other, the end portion of the optical fiber cable inside the connector fixing member 405 and the end portion of the optical fiber cable inside the adapter fixing member 603 are connected to each other.

In the connection device disclosed in U.S. Pat. No. 8,322,932 described above, when a connector fixing member 405 is intended to be inserted into an adapter fixing member 603 such that the connector fixing member 405 is connected to the adapter fixing member 603, a protruding portion 702 of the connector fixing member 405 can gyrate while copying any one of slope end surfaces 801A and 801B respectively in two directions.

Therefore, there is concern that the protruding portion 702 of the connector fixing member 405 bumps into a sharp distal portion of the slope end surfaces 801A and 801B, respectively in two directions, is drawn into an inner surface side of a guide tube 609 of the adapter fixing member 603, and causes jamming between the protruding portion 702 of the connector fixing member 405 and the adapter fixing member 603.

### SUMMARY

A connection device is disclosed in which information transmission cables can be reliably connected to each other while jamming is prevented from occurring in the connection device, and an imaging apparatus provided with the connection device.

According to the present disclosure, a connection device is disclosed, which connects an end portion of a first information transmission cable and an end portion of a second information transmission cable to each other. The connection device includes a connection section that holds a tubular connector fixing member holding the first information transmission cable and having a projection portion on an outer peripheral surface, and a tubular adapter fixing member that holds the second information transmission cable and connects the end portion of the first information transmission cable and the end portion of the second information transmission cable to each other in accordance with insertion of the connector fixing member. The adapter fixing member internally has a guide member. The guide member is provided with a helical guide surface formed in one direction in order to perform an operation in which the projection portion of the connector fixing member is guided in a manner of gyrating around a central axis of the first information transmission cable and the second information transmission cable, when the connector fixing member is inserted.

In the configuration, in the connection device according to the present disclosure, when the connector fixing member is inserted into the guide member in order to connect the end portion of the first information transmission cable and the end portion of the second information transmission cable to each other, the projection portion of the connector fixing member can be guided in a manner of gyrating along the helical guide surface formed in one direction in the guide member without confusing a gyratory direction. That is, the projection portion of the connector fixing member can be reliably guided in a manner of gyrating in only one direction along the helical guide surface formed in one direction in the guide member. Accordingly, in the present disclosure, the connector fixing member and the guide member can be smoothly coupled to each other, and the information transmission cables can be reliably connected to each other while jamming is prevented from occurring in the connection device.

In contrast, in a case of a structure in the known art, the connector fixing member gyrates in any direction of slope end surfaces respectively in two directions. Since there are two slope surfaces, the angle of a sharp distal portion is significant, and there is concern that jamming may occur in the connection device.

In accordance with an exemplary embodiment, it can be preferable that an inner surface of the adapter fixing member is provided with a slope restraining projection portion which restrains the connector fixing member from sloping with respect to the adapter fixing member when the connector fixing member is inserted into the guide member.

In the configuration, even though a force that tends to cause the connector fixing member to slope tends to be generated when the connector fixing member is intended to be inserted in the inner surface of the adapter fixing member, the projection portion causes a force that restrains sloping to act on the connector fixing member. Therefore, the projection portion restrains a force that tends to cause the connector fixing member to slope from being generated, and thus, a phenomenon can be prevented in which the projection portion of the connector fixing member is drawn into an inner surface of the guide member and causes jamming therein.

In accordance with an exemplary embodiment, it can be preferable that an outer peripheral surface of the connection section is provided with a projection portion. It can be preferable that the connection device further includes a connection holding body that internally accommodates the adapter fixing member. It can be preferable that the connection holding body is provided with a helical guide groove portion, which guides the projection portion in a manner of gyrating around the central axis. It can be preferable that before an operation in which the projection portion of the connector fixing member is guided by the helical guide surface in a gyrating manner starts, an operation in which the projection portion of the connection section is guided by the helical guide groove portion in a gyrating manner is configured to start.

In the configuration, at the point of time the projection portion of the connection section enters the helical guide groove portion, the projection portion of the connector fixing member has not yet bumped into a helical vertex portion of the helical guide surface of the guide member, and a gyration guiding operation for the projection portion of the connection section is performed prior thereto along the helical guide groove portion from a leading end portion of the helical guide groove portion and proceeds. When the projection portion hits the helical guide surface of the guide member, the projection portion is guided by the helical guide surface in a gyrating manner. Accordingly, the projection portion can be smoothly guided by the helical guide surface, and the end portions of the information transmission cables can be reliably and smoothly connected to each other.

In accordance with an exemplary embodiment, it can be preferable that a distal portion on the outer peripheral surface of the connector fixing member is provided with a guide projection, which guides the connector fixing member in a manner of gyrating around the central axis by coming into contact with the helical guide surface of the guide member prior to the projection portion when the connector fixing member is inserted into the guide member.

In the configuration, when the connector fixing member is inserted, the guide projection hits the helical vertex portion of the helical guide surface prior to the projection portion. Therefore, rotation along the helical guide surface of the guide member can be induced such that the projection portion can be guided accurately into the guide member along the helical guide surface by the guiding projection. That is, before the guiding projection portion hits the helical vertex portion, the guide projection bumps into the helical vertex portion. Therefore, the projection portion can avoid hitting the helical vertex portion. Accordingly, the projection portion can be caused to smoothly copy the helical guide surface from the position where the projection portion has avoided the helical vertex portion of the helical guide surface.

In accordance with an exemplary embodiment, it can be preferable that the first information transmission cable has an imaging core, and the imaging core generates an optical signal or an acoustic signal.

In the configuration, the imaging core can adopt any one of the optical signal and the acoustic signal in order to form an image.

An imaging apparatus is disclosed, which includes the connection device according to the present disclosure. The imaging apparatus includes a catheter unit that has a connection section; a drive device that has a connection holding body, emits a signal to a target through a first information transmission cable and a second information transmission cable, and obtains a signal from the target through the first information transmission cable and the second information transmission cable; and an operation control apparatus that forms an image of the target in accordance with a signal which is generated based on the signal from the target and is sent from the drive device.

In the configuration, in the imaging apparatus provided with the connection device according to the present disclosure, when the connector fixing member is inserted into the guide member in order to connect the end portion of the first information transmission cable and the end portion of the second information transmission cable to each other, the projection portion of the connector fixing member can be guided in a manner of gyrating along the helical guide surface formed in one direction in the guide member without confusing a gyratory direction. That is, the projection portion of the connector fixing member can be reliably guided in a manner of gyrating in only one direction along the helical guide surface formed in one direction in the guide member. Accordingly, in the present disclosure, the connector fixing member and the guide member can be smoothly coupled to each other, and the information transmission cables can be reliably connected to each other while jamming is prevented from occurring in the connection device.

A connection device is disclosed, which connects an end portion of a first information transmission cable and an end portion of a second information transmission cable to each other, the connection device comprising: a tubular connector configured to hold the first information transmission cable and having a projection portion on an outer peripheral surface; and a tubular adapter configured to hold the second information transmission cable and connects the end portion of the first information transmission cable and the end portion of the second information transmission cable to each other in accordance with insertion of the tubular connector, the tubular adapter internally having a guide member, and wherein the guide member has a helical guide surface formed in one direction in order to perform an operation in which the projection portion of the tubular connector is guided in a manner of gyrating around a central axis of the first information transmission cable and the second information transmission cable, when the tubular connector is inserted.

According to the present disclosure, the connection device is disclosed in which the information transmission cables can be reliably connected to each other while jamming is prevented from occurring in the connection device, and the imaging apparatus provided with the connection device.

### BRIEF DESCRIPTION OF THE DRAWINGS

FIG. 1 is a perspective view illustrating the appearance of an optical coherence tomography apparatus, which serves as an imaging apparatus and is provided with a connection device of a first embodiment of the present disclosure.

FIG. 2 is a perspective view illustrating the appearance of a scanner & pull-back unit (motor drive unit: MDU) illustrated in FIG. 1.

FIG. 3 is a view illustrating a structural example of a catheter unit illustrated in FIG. 1.

FIG. 4 is a cross-sectional view illustrating a structural example of a regional portion BB of a connection section of a drive shaft connector and a regional portion CC of a catheter connection section of the scanner & pull-back unit illustrated in FIG. 3.

FIGS. 5A-5D are views illustrating an example of a shape of a connector fixing member.

FIGS. 6A-6D are perspective views illustrating the connector fixing member illustrated in FIGS. 5A-5D, and a guide member of an adapter fixing member.

FIGS. 7A and 7B are views illustrating a state where the guide member of the adapter fixing member relatively rotates.

FIGS. 8A and 8B are views illustrating a structural example of connecting and fixing the connection section of the drive shaft connector and the catheter connection section of the scanner & pull-back unit to each other, and the view illustrates a state immediately before being fixed.

FIGS. 9A and 9B are views illustrating a state in the middle of an operation of fixing the connection section of the drive shaft connector and the catheter connection section of the scanner & pull-back unit to each other.

FIGS. 10A and 10B are views illustrating a state where the connection section of the drive shaft connector and the catheter connection section of the scanner & pull-back unit are fixed to each other.

FIG. 11B is a view illustrating the first embodiment of the present disclosure and a comparative example as shown in FIG. 11A, which are contrasted with each other.

FIGS. 12A and 12B are views describing a relationship of the first embodiment of the present disclosure with respect to that in FIGS. 11A and 11B.

FIG. 13 is a perspective view illustrating a second embodiment of the present disclosure.

FIG. 14 is a side view including a cross section illustrating the second embodiment of the present disclosure.

FIG. 15 is a perspective view illustrating a third embodiment of the present disclosure.

FIGS. 16A and 16B are cross-sectional views illustrating the third embodiment of the present disclosure.

FIG. 17 is a perspective view illustrating a fourth embodiment of the present disclosure.

FIGS. 18A and 18B are side views illustrating the fourth embodiment of the present disclosure.

FIG. 19 is a cross-sectional view illustrating a fifth embodiment of the present disclosure.

### DESCRIPTION OF EMBODIMENTS

Hereinafter, description will be given regarding specified configurations of the present disclosure and operation effects, which are realized based on each of the configurations. In this case, in order to make the description easy to understand, the reference signs used for illustrating the below-described embodiments are applied to the corresponding configurations. However, the present disclosure is not limited to the detailed configurations of the embodiments to which the reference signs are applied.

Hereinafter, preferable embodiments of the present disclosure will be described in detail with reference to the drawings.

Note that, the embodiments described below are specific examples suitable for the present disclosure, and various types of technically preferable limitation are applied to the embodiments. However, the scope of the present disclosure is not limited by the forms of the limitation unless there is disclosure particularly limiting the present disclosure in the description below.

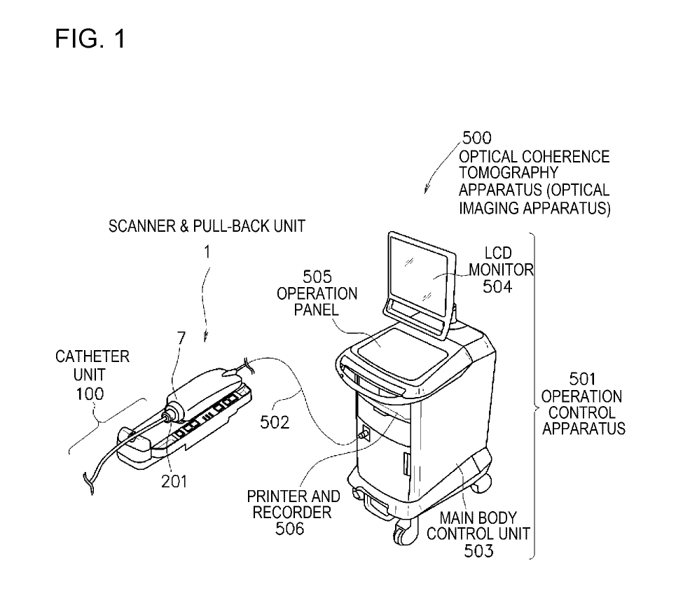

FIG. 1 is a perspective view illustrating the appearance of an optical coherence tomography apparatus 500 provided with a connection device of a first embodiment of the present disclosure. The optical coherence tomography apparatus 500 is an example, particularly an optical imaging apparatus among imaging apparatuses.

However, the imaging apparatus according to the present disclosure is not limited to the optical coherence tomography apparatus 500, which forms an image by using optical signals. The imaging apparatus may be a different medical instrument, for example, an intravascular ultrasound imaging system, which forms an image by using ultrasound signals.

As illustrated in FIG. 1, the optical coherence tomography apparatus 500 can include a catheter unit 100 attachably and detachably serving as an optical probe, a scanner & pull-back unit 1, and an operation control apparatus 501. The scanner & pull-back unit 1 and the operation control apparatus 501 are connected to each other through a signal cable 502.

The catheter unit 100 is directly inserted into a blood vessel of a patient, and the state inside the blood vessel is measured by using low coherent light emitted from an optical imaging core. A connection section 201 of the catheter unit 100 is attachably and detachably connected to a catheter connection section 7 of the scanner & pull-back unit 1. The scanner & pull-back unit 1 is also referred to as a motor drive unit (MDU) or a drive device and executes radial scanning of the optical imaging core inside the catheter unit 100.

The operation control apparatus 501 illustrated in FIG. 1 has a function of inputting various types of setting values during optical coherence tomographic diagnosis, a function of processing data obtained through measurement and displaying the data as a cross-sectional image, and the like. The operation control apparatus 501 has a main body control unit 503, an LCD monitor 504, an operation panel 505, and printer and recorder 506. The main body control unit 503 performs processing of the data obtained through measurement and outputs a processing result. The LCD monitor 504 displays the processing result. A user inputs various types of the setting values through the operation panel 505. The printer and recorder 506 print the processing result and store the processing result as data.

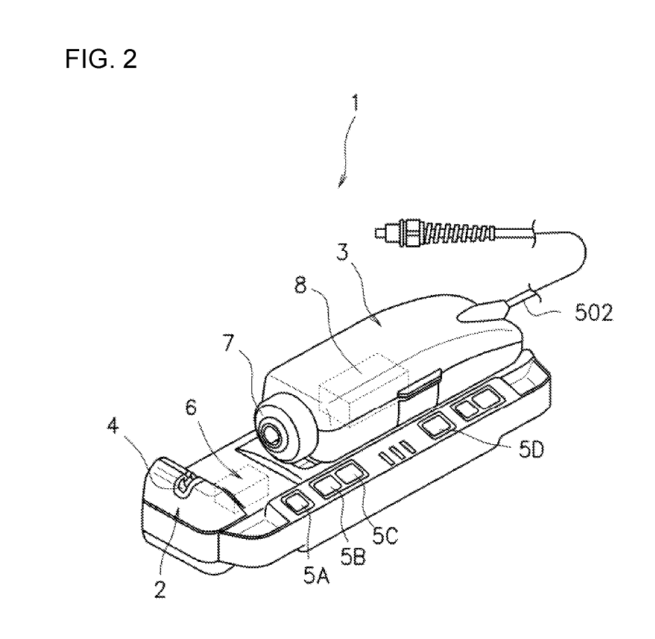

Next, with reference to FIG. 2, an example of the scanner & pull-back unit 1 will be described. FIG. 2 is a perspective view illustrating the appearance of an example of the scanner & pull-back unit 1 illustrated in FIG. 1.

The scanner & pull-back unit 1 illustrated in FIG. 2 has a pull-back unit 2 and a scanner unit 3. The pull-back unit 2 has a catheter clamp 4, operation buttons 5A to 5D, and a pull-back motor 6. The scanner unit 3 has the catheter connection section 7 and a scanner motor 8.

The scanner & pull-back unit 1 illustrated in FIG. 2 emits near infrared light output by a wavelength variable semiconductor laser therein, with respect to a vascular wall through the catheter unit 100 illustrated in FIG. 1. The scanner & pull-back unit 1 changes reflected light received from a target into a signal and sends the signal to the main body control unit 503 through the signal cable 502. The main body control unit 503 acquires an optical coherence tomographic image (OCT image) by performing processing of the obtained signal. For example, the scanner & pull-back unit 1 divides the near infrared light output by the wavelength variable semiconductor laser into two routes for measurement light and reference light. The measurement light is radially emitted to a vascular wall through a designated catheter unit 100 in a rotating manner, and reflected light thereof is acquired through the catheter unit 100. The main body control unit 503 performs Fourier transformation of a coherence signal generated by causing the acquired reflected light and reference light to interfere with each other. The main body control unit 503 obtains reflectance information regarding the depth of the vascular wall and arranges the information in the rotary direction, thereby obtaining an optical coherence tomographic image of the blood vessel.

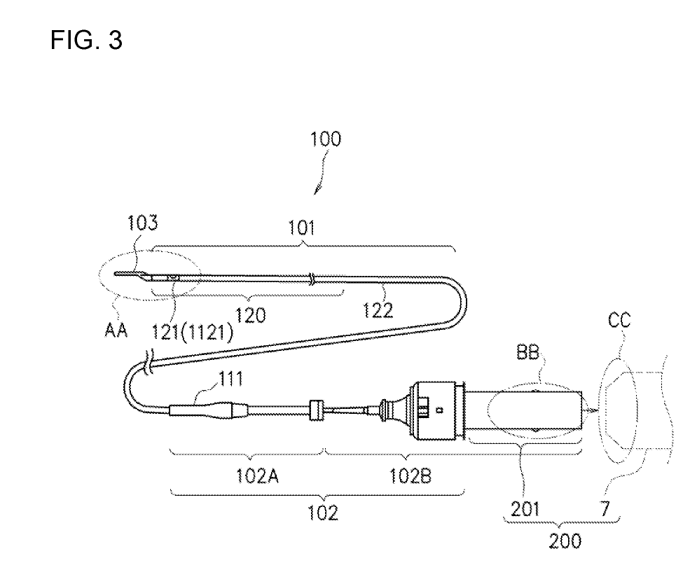

FIG. 3 is a view illustrating a structural example of the catheter unit 100 illustrated in FIG. 1.

The catheter unit 100 illustrated in FIG. 3 has a catheter sheath 101 and a connector portion 102. A distal portion of the catheter sheath 101 is provided with a tube 103 configuring a guide wire lumen. The catheter sheath 101 is continuously formed from the tube 103 to the connector portion 102.

A light transmitting and receiving unit 121 transmitting and receiving measurement light, and an optical fiber cable are accommodated inside the catheter sheath 101 illustrated in FIG. 3. An optical imaging core 120 is disposed substantially throughout the overall length of the catheter sheath 101. The optical imaging core 120 can include a coiled drive shaft 122 for transmitting a drive force which rotates the optical fiber cable.

The connector portion 102 illustrated in FIG. 3 can include a sheath connector 102A and a drive shaft connector 102B. The drive shaft connector 102B is fixed to a proximal portion of the drive shaft 122.

A distal portion of the drive shaft connector 1026 is attachably and detachably connected to a catheter connection holding body 7 of the scanner & pull-back unit 1 illustrated in FIG. 2. In a connection device 200 of the embodiment of the present disclosure, when the connection section 201 of the drive shaft connector 1026 and the catheter connection holding body 7 of the scanner & pull-back unit 1 are connected to each other, an end portion of the optical fiber cable inside the connection section 201 and the end portion of the optical fiber cable inside the catheter connection holding body 7 can be connected and coupled to each other. The optical fiber cables can be reliably and smoothly connected to each other while jamming is prevented when the connection section 201 and the catheter connection holding body 7 are coupled to each other.

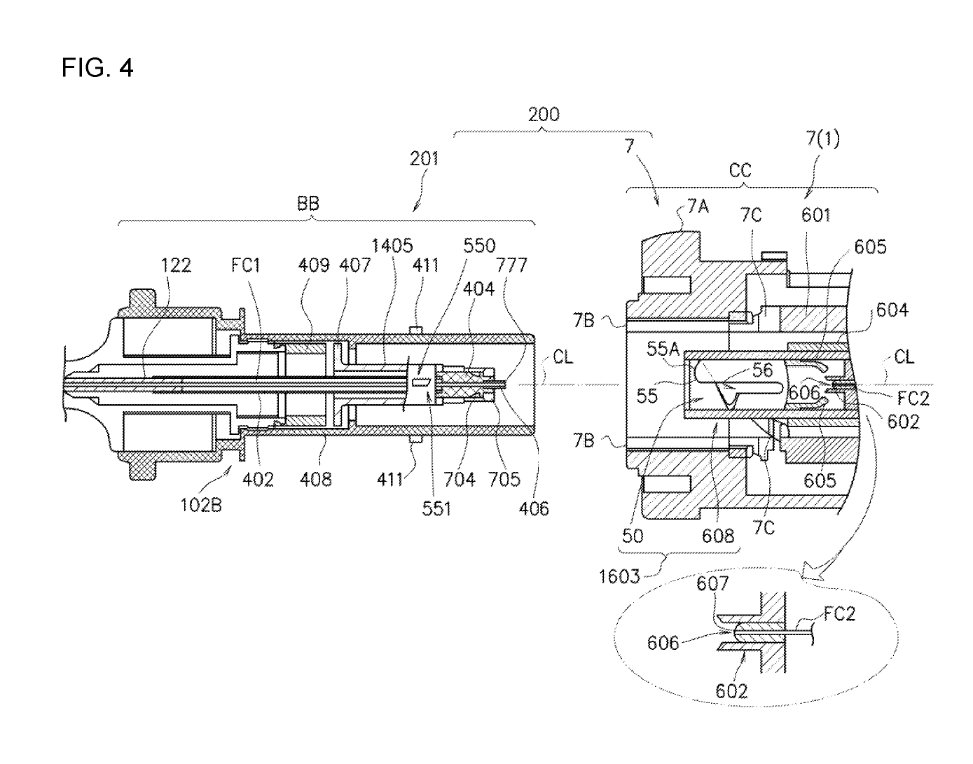

FIG. 4 is a cross-sectional view illustrating a structural example of a regional portion BB of the connection section 201 of the drive shaft connector 102B and a regional portion CC of the catheter connection holding body 7 of the scanner & pull-back unit 1 illustrated in FIG. 3.

The connection section 201 of the drive shaft connector 102B and the catheter connection holding body 7 of the scanner & pull-back unit 1 illustrated in FIG. 4 configure the connection device 200 of the embodiment of the present disclosure. In the connection device 200, an end portion 777 of an optical fiber cable FC1 positioned on a central axis CL of the connection section 201 of the drive shaft connector 102B, and an end portion 607 of an optical fiber cable FC2 positioned on the central axis CL of the catheter connection holding body 7 of the scanner & pull-back unit 1 can be connected to each other by being brought into contact with each other.

The connection section 201 of the drive shaft connector 102B is also referred to as a connector, and the catheter connection holding body 7 of the scanner & pull-back unit 1 can be referred to as an adapter for receiving the connection section 201 which is the connector. For example, the optical fiber cables FC1 and FC2 on one side and the other side are single mode optical fiber cables and each of which is configured to include a core for transmitting light, and a clad having a refractive index slightly smaller than that of the core. A resin coating material can be provided on the outer peripheral surface of the clad.

First, the structural example of the connection section 201 of the drive shaft connector 102B illustrated in FIG. 4 will be described. A connector member 404 for the optical fiber cable FC1 is joined to the drive shaft 122 via a connection pipe 402. A connector fixing member 1405 is a cylindrical member and has a disk-shaped flange 407 at an end portion thereof. The flange 407 is formed at the end portion of the connector fixing member 1405. The connector member 404 is fixed to the inside of the cylindrical connector fixing member 1405. The connector fixing member 1405 is rotatably held inside a housing 408 of the drive shaft connector 1026. A pair of projection portions 411 are respectively formed at positions opposite to each other on the outer peripheral surface of the housing 408.

As illustrated in FIG. 4, a ferrule 406 is provided at the distal end of the connector member 404, and the ferrule 406 is fixed to the end portion of the optical fiber cable FC1. A protruding portion 704 and a round projection 705 are formed at the distal portion of the connector member 404. In order to prevent noise from being generated due to reflection of light on the end surface, the end portion 777 of the optical fiber cable FC1 is subjected to APC-type processing in which a slope angle is formed with respect to a traveling direction of light.

As illustrated in FIG. 4, inside the housing 408, an elastic member 409 is disposed at a position in the vicinity of the flange 407 so as to be able to be in contact therewith. The elastic member 409 pressurizes the flange 407 when the connection section 201 of the drive shaft connector 1026 and the catheter connection holding body 7 of the scanner & pull-back unit 1 are connected to each other, thereby facilitating the operation of connecting the end portion 777 of the optical fiber cable FC1 and the end portion 607 of the optical fiber cable FC2 to each other. After the end portion 777 of the optical fiber cable FC1 and the end portion 607 of the optical fiber cable FC2 are connected (coupled) to each other, the elastic member 409 and the flange 407 are in a non-contact state. Accordingly, the inner members can be prevented from being damaged or deformed during the internal driving. For example, the elastic member 409 can be synthetic rubber, a metal spring, or silicon rubber having low viscosity.

Next, the structural example of the catheter connection holding body 7 of the scanner & pull-back unit 1 illustrated in FIG. 4 will be described.

A housing 601 illustrated in FIG. 4 is fixed to the inside of a head portion 7A of the catheter connection holding body 7. The housing 408 of the drive shaft connector 1026 is fitted to the inner surface of the housing 601 when the connectors are connected to each other. The head portion 7A internally has a groove portion entrance 7B. The housing 601 is provided with a pair of groove portions 7C leading from the groove portion entrance 7B of the head portion 7A. The pair of groove portions 7C can receive the pair of projection portions 411 of the housing 408.

An adapter member 602 illustrated in FIG. 4 is a portion to be coupled to the connector member 404 and is held so as to be relatively rotatable with respect to the housing 601. An adapter fixing member 1603 is a cylindrical member and the adapter member 602 is fixed to the inside thereof so as not to be relatively rotatable. That is, the adapter member 602 and the adapter fixing member 1603 are integrated with each other. The adapter fixing member 1603 performs positioning of the connector member 404 in the circumferential direction in association with the connector fixing member 1405 while being coupled to the connector member 404.

The adapter fixing member 1603 is joined to a drive force relay pipe 604. A drive force of the rotary driving scanner motor 8 illustrated in FIG. 2 is sent to the adapter fixing member 1603 via the drive force relay pipe 604 and is transmitted to the drive shaft 122 after being coupled to the connector member 404. As the adapter fixing member 1603, a 0 dB-attenuator (0 dB-ATT) is employed. However, the adapter fixing member 1603 is not limited thereto.

As illustrated in FIG. 4, a pair of claw portions 605 are formed on the inner surface of the adapter fixing member 1603. The pair of claw portions 605 engage with the connector member 404, thereby causing the connector member 404 and the adapter member 602 to be rigidly integrated with each other in an attachable and detachable manner. A female-type hole 606 receiving the ferrule 406 of the connector member 404 is formed in the adapter member 602, and the end portion 607 of the optical fiber cable FC2 subjected to APC-type processing is fixed to the inner side of the hole 606.

The adapter fixing member 1603 illustrated in FIG. 4 has an outer tubular protective tube 608 and an inner tubular guide member 50. The guide member 50 is fixed to the inner surface of the protective tube 608 or is formed so as to be integrated therewith.

When the connection section 201 of the drive shaft connector 1026 and the catheter connection holding body 7 of the scanner & pull-back unit 1 are joined to each other, the guide member 50 guides the connector fixing member 1405 while relatively gyrating around the central axis CL. Accordingly, the guide member 50 plays a role of positioning around the central axis CL in a gyratory direction by reliably and smoothly guiding the connector fixing member 1405 into the adapter fixing member 1603.

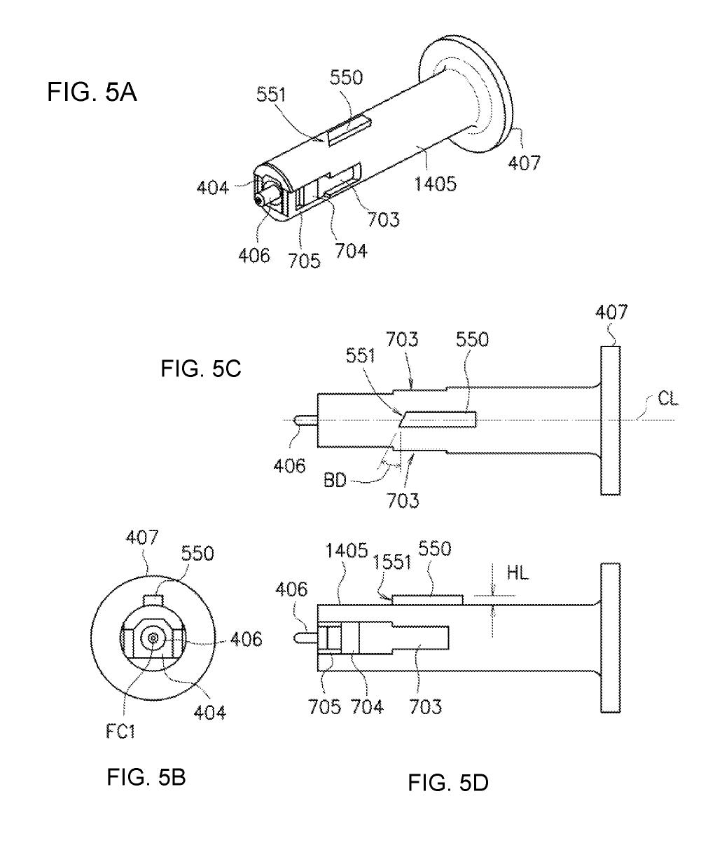

Next, with reference to FIGS. 5A-5D, a shape of the connector fixing member 1405 illustrated in FIG. 4 will be further described. FIGS. 5A-5D illustrate a shape of the connector fixing member 1405.

As described above, the connector fixing member 1405 has the flange 407, symmetrical slits 703 and 703 on the right and left, and one projection portion 550. The connector member 404 is disposed inside the connector fixing member 1405, and the ferrule 406 protrudes from the connector member 404. The outer peripheral surface of the connector fixing member 1405 is provided with the projection member (also referred to as the guide key) 550.

The projection portion 550 is formed so as to be parallel to the central axis CL of the connector fixing member 1405, and the front end portion of the projection portion 550 forms one slope portion 551. As illustrated in FIG. 5C, the one slope portion 551 is formed so as to have a slope angle BD (acute angle) with respect to a line orthogonal to the central axis CL. The projection portion 550 is formed at a position separated from each of the slits 703 and 703 on the right and left by 90 degrees of angle around the central axis CL in the circumferential direction.

Moreover, as illustrated in FIG. 5D, a forming height HL of the projection portion 550 is highly set in order to prevent an occurrence of a state where the projection portion 550 is drawn into the inner surface side of the guide member 50 and causes jamming therein. For example, the forming height HL is set so as to be the same as or greater than the thickness of the guide member 50.

In each of the slits 703, the outer peripheral surface of the connector member 404 is partially exposed. The protruding portion 704 and the round projection 705 are formed on the outer peripheral surface of the connector member 404. The protruding portion 704 and the round projection 705 are positioned inside the slit 703.

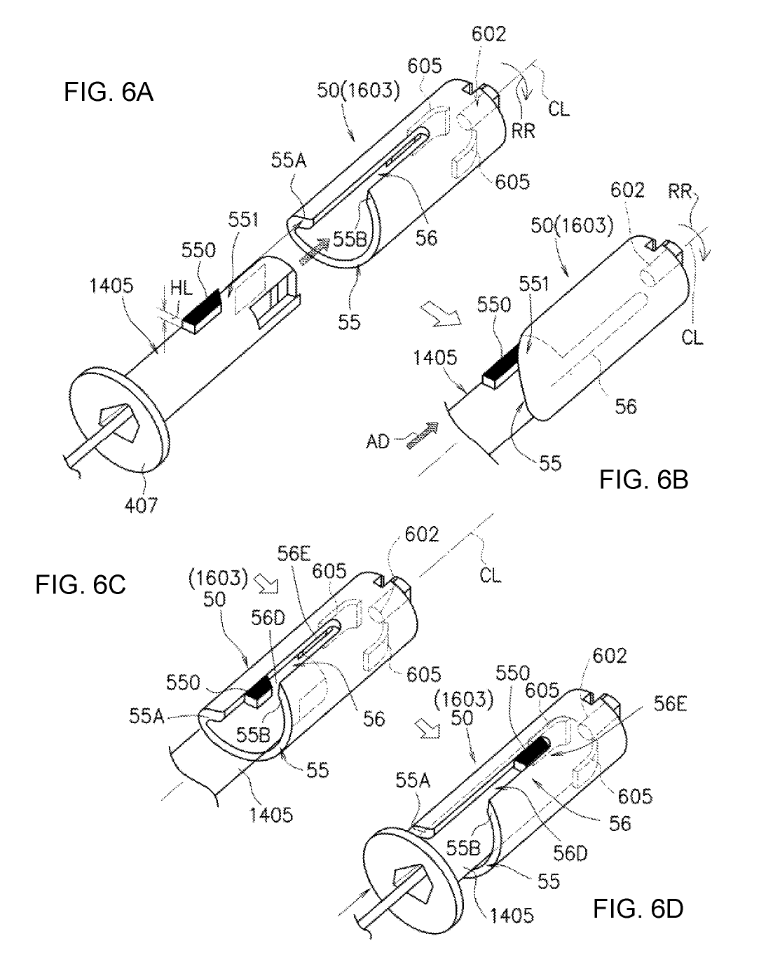

Next, with reference to FIG. 6A-6D, an example of a shape of the guide member 50 of the adapter fixing member 1603 illustrated in FIG. 4 will be described. FIGS. 6A-6D are perspective views illustrating the connector fixing member 1405 illustrated in FIG. 4, and the guide member 50 of the adapter fixing member 1603. In FIGS. 6A-6D, in order to make the view easy to understand, illustration of the protective tube 608 illustrated in FIG. 4 is omitted, and only the guide member 50 of the adapter fixing member 1603 is illustrated.

In FIGS. 6A and 6B, it can be preferable that the guide member 50 of the adapter fixing member 1603 is rotated on the guide member 50 side of the adapter fixing member 1603 in accordance with driving of the motor (the scanner motor 8 illustrated in FIG. 2) at a low speed, and the guide member 50 of the adapter fixing member 1603 and the connector fixing member 1405 are relatively rotated in a gyratory direction RR by an angle approximately a little less than, for example, 360 degrees. Accordingly, as illustrated in FIGS. 6C and 6D, the guide member 50 of the adapter fixing member 1603 and the connector fixing member 1405 are positioned with respect to the gyratory direction RR and the central axis CL, thereby being connected to each other.

First, as illustrated in FIG. 6A, the guide member 50 is a cylindrical member. The guide member 50 has a helical guide surface 55 and a guide groove portion 56. The helical guide surface 55 is formed so as to slope along one direction around the central axis CL from a helical vertex portion 55A to a helical rear end portion 55B and is smoothly formed at a predetermined slope angle, for example, a slope angle of 15 degrees. The guide groove portion 56 is formed so as to be parallel to the central axis CL. The groove width of the guide groove portion 56 is formed by a line extending in a direction of the central axis CL from the helical vertex portion 55A, and a line extending from the helical rear end portion 55B. That is, the helical vertex portion 55A and the guide groove portion 56 are adjacent to each other, and the low point of the helix and the guide groove portion 56 are also adjacent to each other. In accordance with an exemplary embodiment, the guide groove portion 56 is positioned between the helical vertex portion 55A and the low point of the helix. The slope angle BD of one helical guide surface 55 in the illustrated example in FIG. 6A can be set to 15 degrees as an example. However, without being limited thereto, for example, the slope angle BD may be set to 30 degrees.

The slope portion 551 of the projection portion 550 is formed in accordance with the slope angle of the helical guide surface 55. Accordingly, the slope portion 551 of the projection portion 550 is smoothly guided along the helical guide surface 55 from the helical vertex portion 55A to the helical rear end portion 55B. Moreover, the projection portion 550 can be introduced into the guide groove portion 56.

In addition, as illustrated in FIG. 4, in consideration of the safety, the protective tube 608 covers the guide member 50 such that even though the helical vertex portion 55A of the guide surface 55 is sharp, a user's finger by no means touches the helical vertex portion 55A. Moreover, since the protective tube 608 is provided, the helical vertex portion 55A can be prevented from hitting a certain object and being damaged. In addition, the protective tube 608 functions to guide the connector fixing member 1405 when the connector fixing member 1405 is inserted along the central axis CL.

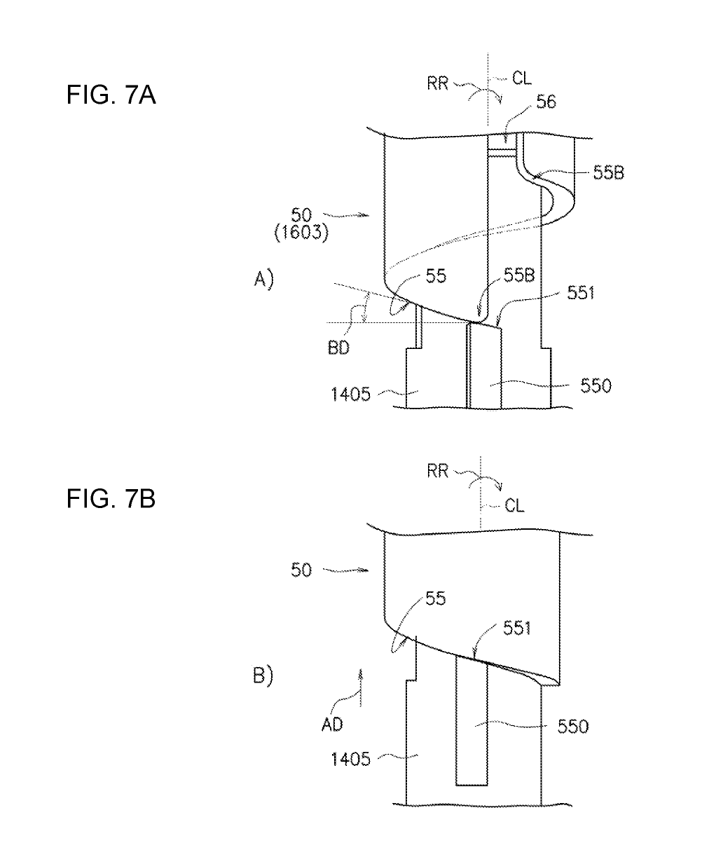

Next, with reference to FIGS. 6A-6D, 7A, and 7B, description will be given regarding an example of a connection operation performed along the gyratory direction RR in which the connector fixing member 1405 and the guide member 50 of the adapter fixing member 1603 are coupled to each other. FIGS. 7A and 7B illustrate a state where the guide member 50 of the adapter fixing member 1603 rotates. In this case, it can be preferable that the guide member 50 is rotated by driving the motor (the scanner motor 8 illustrated in FIG. 2) at a low speed.

When a user holds the connection section 201 of the drive shaft connector 102B illustrated in FIG. 3, by hand and performs connection by causing the connection section 201 thereof to be fitted to the catheter connection holding body 7 of the scanner & pull-back unit 1, as illustrated in FIG. 6A, the distal portion of the connector fixing member 1405 approaches the guide member 50 of the adapter fixing member 1603. Accordingly, as illustrated in FIG. 7A, the slope portion 551 of the projection portion 550 of the connector fixing member 1405 bumps into the helical vertex portion 55A of the guide surface 55.

In response to driving of the scanner motor 8 illustrated in FIG. 2, the guide member 50 of the adapter fixing member 1603 rotates around the central axis CL in the gyratory RR direction at a low speed. Therefore, as illustrated in FIGS. 6B and 7B, the slope portion 551 of the projection portion 550 is guided along the helical rear end portion 55B from the helical vertex portion 55A of the guide surface 55. Accordingly, the connector fixing member 1405 advances in an insertion direction AD.

Then, gyrating of the guide member 50 of the adapter fixing member 1603 in the gyratory direction RR proceeds, and as illustrated in FIG. 6C, the slope portion 551 of the projection portion 550 arrives at a position of a distal portion 56D of the guide groove portion 56 from the helical rear end portion 55B. Here, the user further presses the connection section 201 of the drive shaft connector 102B in FIG. 3 by hand, and as illustrated in FIG. 6D, the projection portion 550 is guided to an inner end portion 56E of the guide groove portion 56 and bumps into the inner end portion 56E.

Accordingly, the pair of claw portions 605 of the adapter fixing member 1603 illustrated in FIG. 4 individually engage with the protruding portion 704 of the connector member 404, and the ferrule 406 is fitted to the adapter member 602. The end portion of the optical fiber cable FC1 and the end portion of the optical fiber cable FC2 can be reliably and smoothly coupled to each other.

In this manner, when the user holds the connection section 201 of the drive shaft connector 102B illustrated in FIG. 3, by hand and mounts the connection section 201 thereof in the catheter connection holding body 7 of the scanner & pull-back unit 1, in regard to the below-described point, the end portion 777 of the optical fiber cable FC1 and the end portion 607 of the optical fiber cable FC2 illustrated in FIG. 4 can be reliably and smoothly connected to each other.

As illustrated in FIGS. 6A-6D, 7A, and 7B, one helical guide surface 55 is sloped along only one direction and is smoothly formed around the central axis CL from the helical vertex portion 55A to the helical rear end portion 55B in a continuously helical manner. The slope portion 551 of the projection portion 550 is formed in accordance with the slope angle BD of the helical guide surface 55. Accordingly, the slope portion 551 of the projection portion 550 is smoothly guided along the helical guide surface 55 from the helical vertex portion 55A to the helical rear end portion 55B, and thus, the slope portion 551 can be introduced into the guide groove portion 56.

In addition, as illustrated in FIGS. 6A-6D, the forming height HL of the projection portion 550 is highly set in order to prevent an occurrence of a state where the projection portion 550 is drawn into the inner surface side of the guide member 50 and is jammed therein. Accordingly, the projection portion 550 can avoid a state of being drawn into the inner surface of the guide member 50 and causing jamming therein. If the projection member is drawn into the inner surface of the guide member and causes jamming therein, there is concern that the guide member, the protective member, and peripheral portions thereof may be damaged. When damage occurs, there is a need to perform a repair, and the scanner & pull-back unit 1 illustrated in FIG. 1 cannot be used any longer.

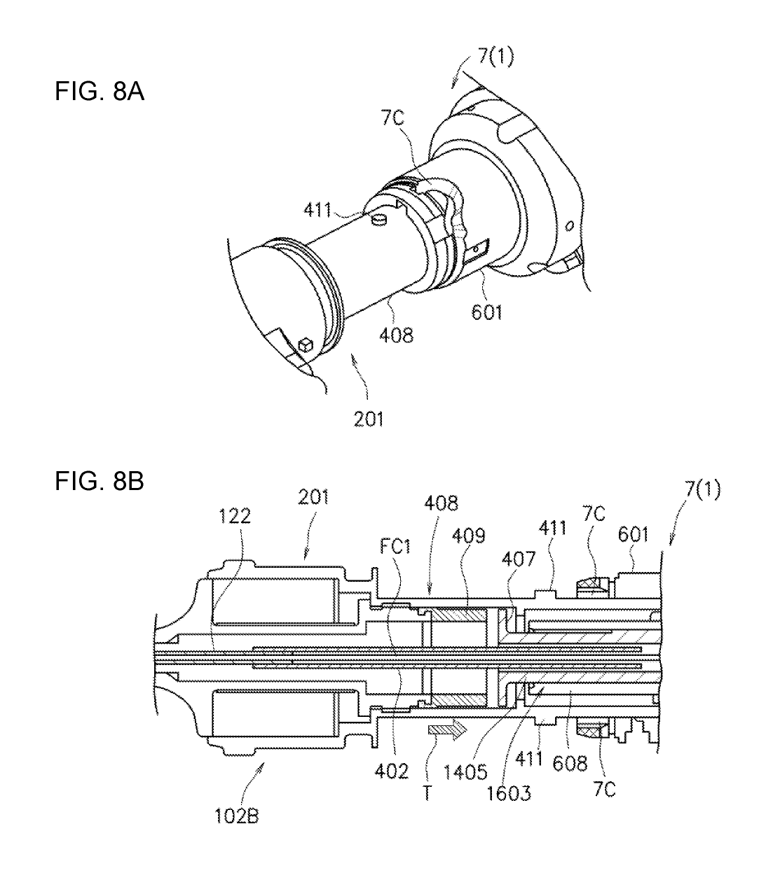

FIGS. 8A to 10B illustrate a structural example of connecting and fixing the connection section 201 of the drive shaft connector 102B and the catheter connection holding body 7 of the scanner & pull-back unit 1 to each other. Here, with reference to FIGS. 8A to 10B, description will be given regarding an example of a procedure of connecting and fixing the connection section 201 of the drive shaft connector 102B and the catheter connection holding body 7 of the scanner & pull-back unit 1 to each other. The structure of connecting and fixing thereof is not limited to the illustrated example, and an arbitrary structure can be employed.

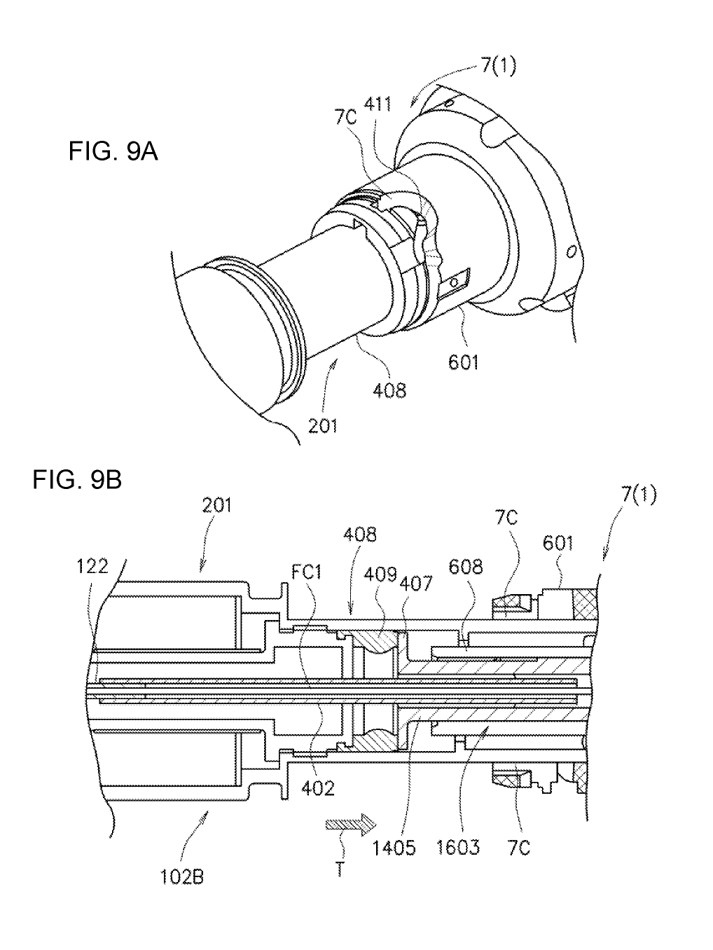

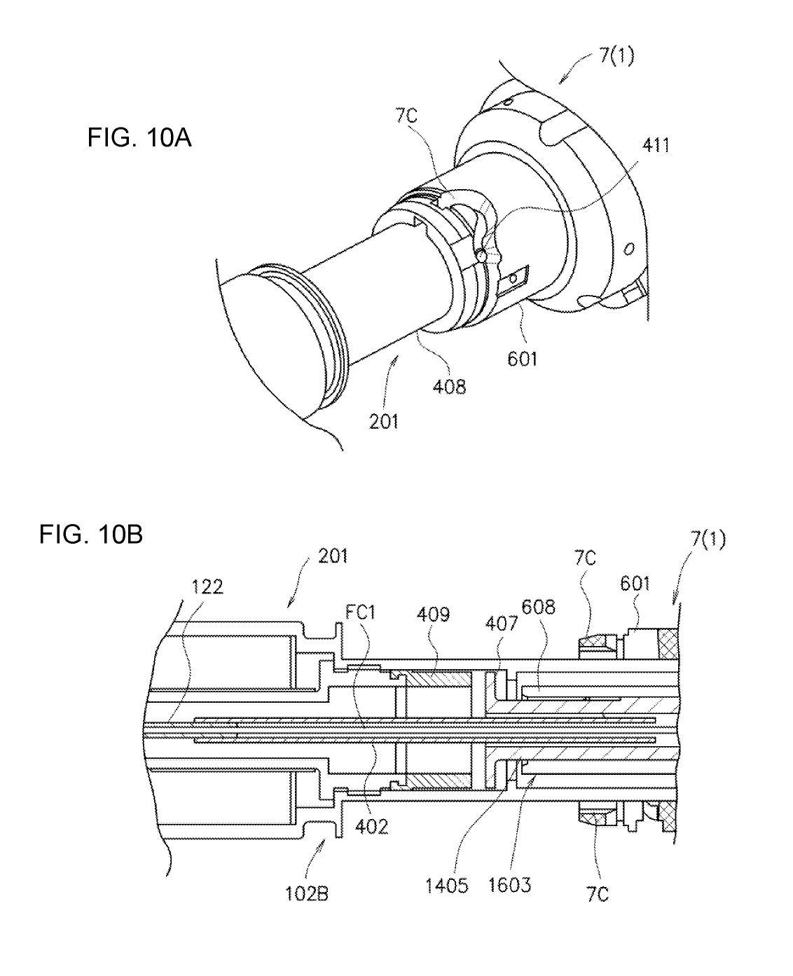

FIGS. 8A and 8B are views illustrating a state immediately before the connection section 201 of the drive shaft connector 102B and the catheter connection holding body 7 of the scanner & pull-back unit 1 are connected and fixed to each other. FIGS. 9A and 9B are views illustrating a state in the middle of an operation of fixing the connection section 201 of the drive shaft connector 102B and the catheter connection holding body 7 of the scanner & pull-back unit 1 to each other. FIGS. 10A and 10B are views illustrating a state where the connection section 201 of the drive shaft connector 102B and the catheter connection holding body 7 of the scanner & pull-back unit 1 are fixed to each other.

As illustrated in FIGS. 8A and 8B, the pair of projection portions 411 of the connection section 201 are inserted along an insertion direction T with respect to the groove portions 7C of the catheter connection holding body 7. At this time, as illustrated in FIG. 8B, the flange 407 and the elastic member 409 are in a non-contact state.

Next, as illustrated in FIGS. 9A and 9B, when the housing 408 of the connection section 201 is further inserted into the insertion direction T and the projection portions 411 are respectively positioned at the deepest positions in the groove portions 7C, as illustrated in FIG. 9B, the flange 407 pressurizes the elastic member 409 and the elastic member 409 is compressed. At this time, the end portion 777 of the optical fiber cable FC1 and the end portion 607 of the optical fiber cable FC2 illustrated in FIG. 4 are connected to each other as described above.

Moreover, as illustrated in FIGS. 10A and 10B, when the projection portions 411 are caused to slide along the groove portions 7C, the projection portions 411 advance in the gyratory direction perpendicular to the insertion direction T, and the projection portions 411 stop at a trailing end portion of the groove portion 7C. At this time, as illustrated in FIG. 10B, the flange 407 and the elastic member 409 are separated from each other and return to the non-contact state.

In this manner, the connection section 201 of the drive shaft connector 102B and the connection holding body 7 of the scanner & pull-back unit 1 can be connected and fixed to each other.

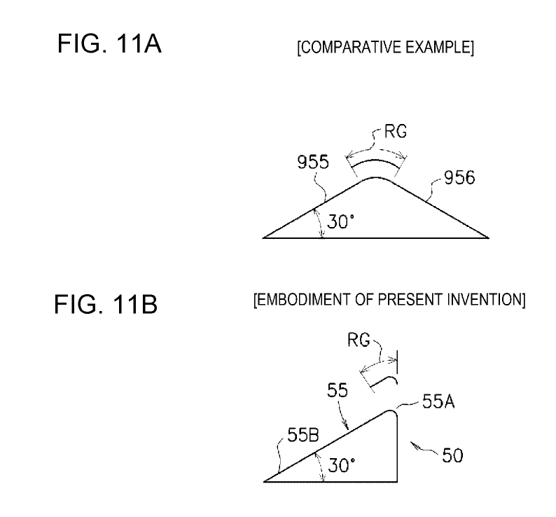

Here, with reference to FIGS. 11A and 11B, the first embodiment of the present disclosure and a comparative example, which is beyond the scope of the present disclosure will be described.

In the comparative example illustrated in FIG. 11A, two sloped guide surfaces 955 and 956 are formed. In the guide surfaces 955 and 956, a range RG in which a connector fixing member may cause jamming is wide.

In contrast, in the embodiment of the present disclosure illustrated in FIG. 11B, in the guide member 50, since there is one sloped guide surface 55 formed in only one direction, a range RT in the guide surface 55 can be narrower than the range RG.

As described above, one sloped guide surface 55 is employed such that the connector fixing member 1405 can rotate along a direction rotating around the central axis CL. Moreover, the forming height HL of the connector fixing member 1405 is set to be significant as much as possible. Accordingly, the connector fixing member 1405 can be reliably prevented from being drawn into the inner surface side of the guide member 50 and causing jamming therein.

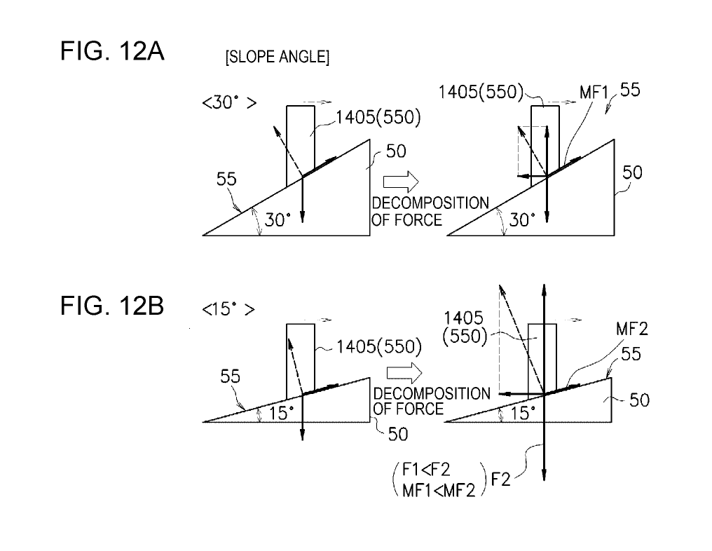

FIGS. 12A and 12B respectively illustrate cases where the slope angle of one guide surface 55 of the guide member 50 can be, for example, 30 degrees and 15 degrees. A force F1 which is illustrated in FIG. 12A, is generated in a case where the slope angle is 30 degrees, and thrusts the catheter (the connector fixing member 1405) can be smaller than a force F2 which is illustrated in FIG. 12B, is generated in a case where the slope angle can be, for example, 15 degrees, and thrusts the catheter (the connector fixing member 1405).

In accordance with an exemplary embodiment, as the slope angle of the one guide surface 55 increases, a force that can guide the catheter (the connector fixing member 1405) can be generated by a smaller force of thrusting the catheter (the connector fixing member 1405), and thus, as seen from the comparison between a frictional force MF1 and a frictional force MF2, a generated frictional force can also be reduced. Therefore, when the slope angle is significant, connecting work of connecting the end portion 777 of the optical fiber cable FC1 and the end portion 607 of the optical fiber cable FC2 illustrated in FIG. 4 connecting work can be more easily performed.

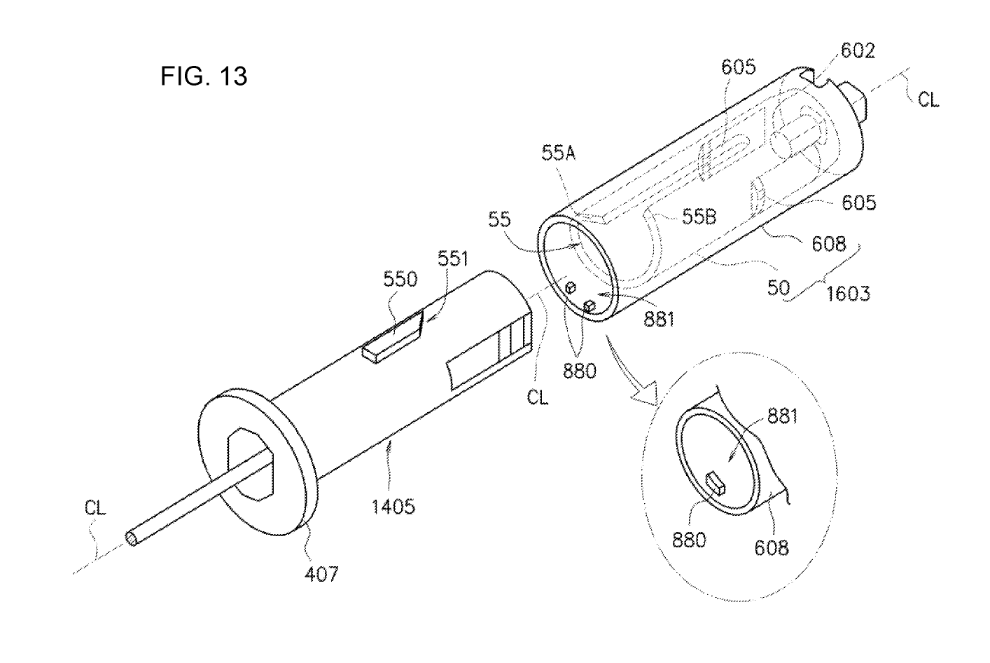

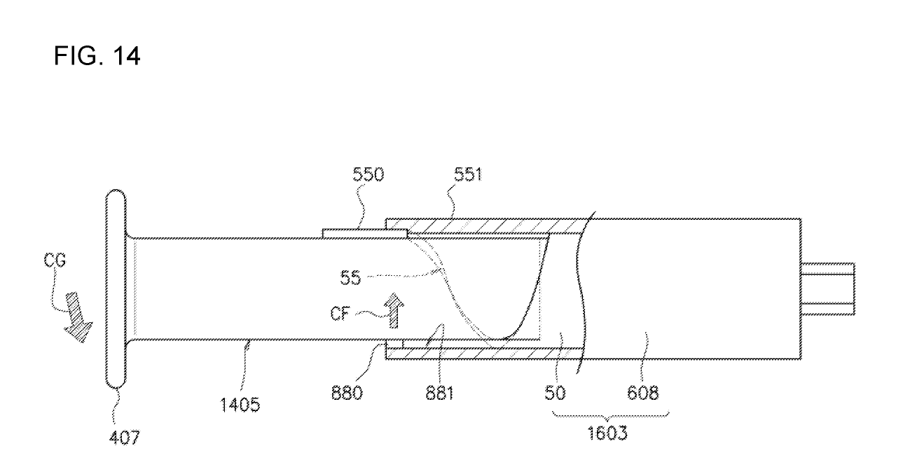

Next, with reference to FIGS. 13 and 14, a second embodiment of the present disclosure will be described.

FIG. 13 is a perspective view illustrating the connector fixing member 1405 of the second embodiment of the present disclosure, and the adapter fixing member 1603. FIG. 14 is a side view having a cross section illustrating a state where the connector fixing member 1405 is inserted into the adapter fixing member 1603.

The structures of the connector fixing member 1405 of the second embodiment of the present disclosure illustrated in FIGS. 13 and 14, and the guide member 50 of the adapter fixing member 1603 are the same as those of the connector fixing member 1405 illustrated in FIGS. 7A and 7B, and the guide member 50 of the adapter fixing member 1603. The adapter fixing member 1603 has the outer tubular protective tube 608 and the inner tubular guide member 50. The guide member 50 is fixed to the inner surface of the protective tube 608 or is formed so as to be integrated therewith.

However, in the second embodiment of the present disclosure, one or a plurality of slope restraining projection portions 880 are additionally provided on the inner surface of the protective tube 608 of the adapter fixing member 1603. The slope restraining projection portion 880 is formed so as to protrude on an inner surface 881 of an opening distal portion of the protective tube 608. The position where the projection portion 880 is formed is a position facing the helical vertex portion 55A of the one guide surface 55, and is a position where the guiding projection portion 550 does not hit the slope restraining projection portion 880 when the connector fixing member 1405 is pulled out from the inside of the protective tube 608. Accordingly, when the connector fixing member 1405 is detached, the connector fixing member 1405 can be smoothly pulled out so as not to hit the projection portion 880 from the inside of the protective tube 608.

As illustrated in FIGS. 13 and 14, on the inner surface of the protective tube 608 of the adapter fixing member 1603, one or the plurality of slope restraining projection portions 880 are provided. As illustrated in FIG. 14, even though a force CG that tends to cause the flange 407 side of the connector fixing member 1405 to slope tends to be generated when the connector fixing member 1405 is intended to be inserted in the inner surface of the protective tube 608 of the adapter fixing member 1603, the projection portion 880 causes a force CF that restrains sloping to act on the connector fixing member 1405.

Therefore, the projection portion 880 restrains the force CG that tends to cause the flange 407 side of the connector fixing member 1405 to slope from being generated, and thus, a phenomenon can be further prevented in which the guiding projection portion 550 of the connector fixing member 1405 is drawn into the inner surface of the guide member 50 and causes jamming therein.

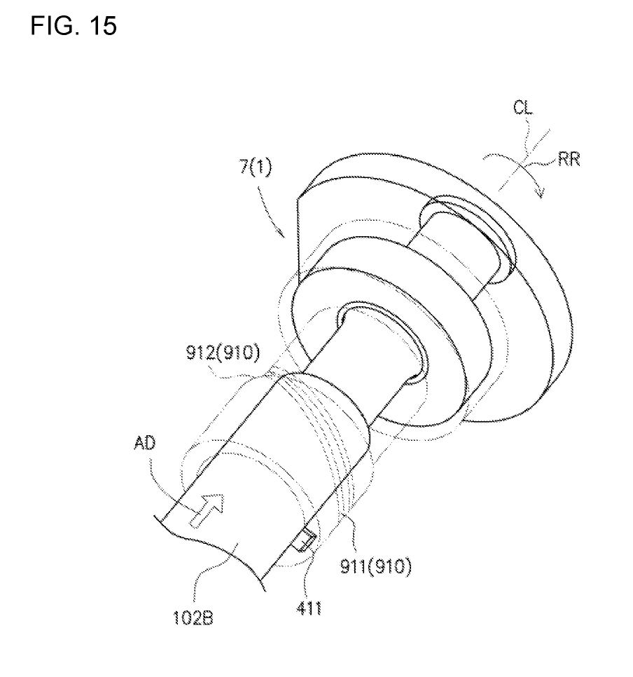

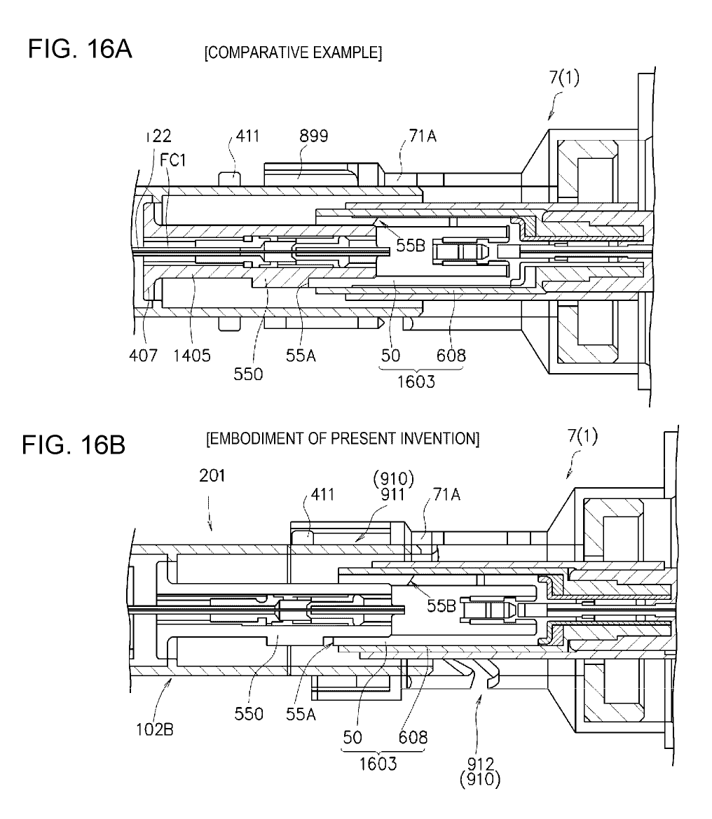

Next, with reference to FIGS. 15 and 16B, a third embodiment of the present disclosure will be described.

FIGS. 15 and 16B illustrate a structural example in which the connection section 201 of the drive shaft connector 102B of the third embodiment of the present disclosure, and the catheter connection holding body 7 of the scanner & pull-back unit 1 are connected and fixed to each other. FIG. 15A illustrates a different structural example for comparison.

As illustrated in FIGS. 15 and 16B, a helical guide groove portion 910 is formed in the catheter connection holding body 7. The helical guide groove portion 910 is formed around the central axis CL in a helical manner and is formed in the same gyratory direction as that of the helical guide surface 55 of the guide member 50. The helical guide groove portion 910 has a leading end portion 911 lead to a trailing end portion 912.

Accordingly, as illustrated in FIG. 16B, at the point of time the projection portions 411 of the connection section 201 of the drive shaft connector 102B enters the leading end portion 911 of the helical guide groove portion 910, the guiding projection portion 550 of the connector fixing member 1405 has not yet bumped into the helical vertex portion 55A of the helical guide surface 55 of the guide member 50, and a gyration guiding operation for the projection portions 411 of the connection section 201 of the drive shaft connector 102B is performed prior thereto along the helical guide groove portion 910 from the leading end portion 911 of the helical guide groove portion 910 and proceeds along the central axis CL. In accordance with an exemplary embodiment, the projection portions 411 of the connection section 201 take the lead and start a gyration guiding operation.

When the guiding projection portion 550 illustrated in FIG. 16B hits a middle portion of the helical guide surface 55 of the guide member 50 (a position avoiding the helical vertex portion 55A), the guiding projection portion 550 is guided by the helical guide surface 55 and starts a gyration guiding operation around the central axis CL.

Accordingly, after the projection portion 411 of the connection section 201 take the lead and start the gyration guiding operation, the guiding projection portion 550 can be smoothly guided in a manner of gyrating from the middle portion of the helical guide surface 55 to the helical rear end portion 55B. Therefore, end portions 777 and 607 of the optical fiber cables FC1 and FC2 can be reliably and smoothly connected to each other.

In contrast, in the comparative example illustrated in FIG. 16A, the guiding projection portion 550 bumps into the helical vertex portion 55A of the helical guide surface 55 of the guide member 50 in advance, and the projection portions 411 of the connection section 201 have not yet entered a helical guide groove portion 899.

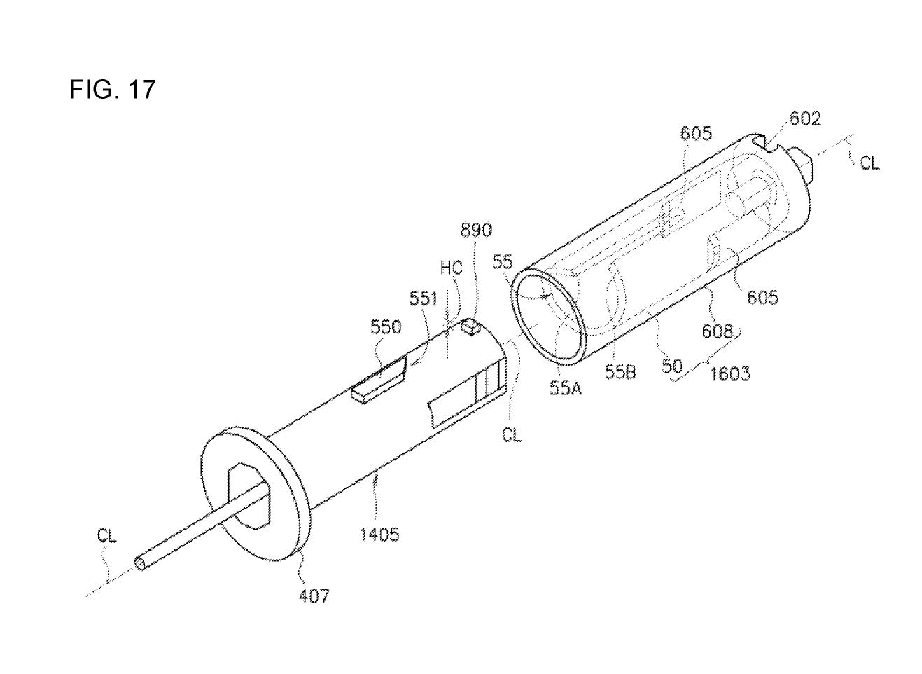

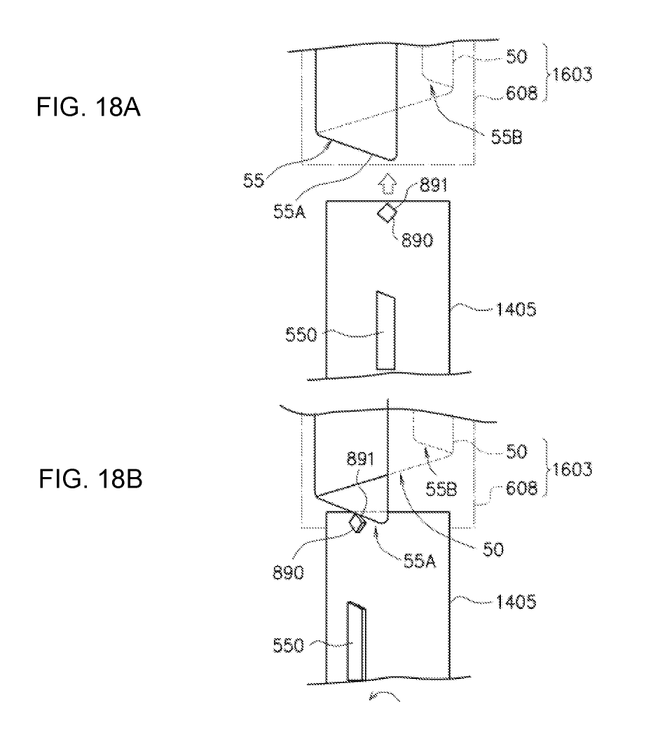

Next, with reference to FIGS. 17, 18A, and 18B, a fourth embodiment of the present disclosure will be described.

FIG. 17 is a perspective view illustrating the connector fixing member 1405 of the fourth embodiment of the present disclosure, and the adapter fixing member 1603. FIGS. 18A and 18B are views illustrating a state where the connector fixing member 1405 is inserted into the adapter fixing member 1603.

The structures of the connector fixing member 1405 illustrated in FIGS. 17, 18A, and 18B, and the guide member 50 of the adapter fixing member 1603 are the same as those of the connector fixing member 1405 illustrated in FIGS. 7A and 7B, and the guide member 50 of the adapter fixing member 1603. The adapter fixing member 1603 has the outer tubular protective tube 608 and the inner tubular guide member 50. The guide member 50 is fixed to the inner surface of the protective tube 608 or is formed so as to be integrated therewith.

However, in the fourth embodiment of the present disclosure, a leading guide projection 890 is additionally formed at a position of the distal portion on the outer peripheral surface of the connector fixing member 1405. The guide projection 890 is formed at a position ahead of the guiding projection portion 550 of the connector fixing member 1405 (distal portion side) along the central axis CL, and the guide projection 890 is positioned in the distal portion so as to be able to take the lead prior to the guiding projection portion 550.

For example, the guide projection 890 is formed so as to have a square shape as illustrated in FIGS. 18A and 18B. However, the shape is not particularly limited. A distal surface 891 of the guide projection 890 is sloped with respect to the central axis CL. A protrusion forming height HC of the guide projection 890 may be the same as or slightly smaller than the thickness of the guide member 50.

Accordingly, when the connector fixing member 1405 is inserted into the tubular protective tube 608 of the adapter fixing member 1603, the guide projection 890 takes the lead prior to the guiding projection portion 550 and hits the helical vertex portion 55A of the helical guide surface 55. Therefore, rotation along the helical guide surface 55 of the guide member 50 can be induced. In accordance with an exemplary embodiment, before the guiding projection portion 550 hits the helical vertex portion 55A, the guide projection 890 bumps into the helical vertex portion 55A. Therefore, the guiding projection portion 550 can avoid directly hitting the helical vertex portion 55A. Accordingly, the guiding projection portion 550 can be caused to smoothly copy the helical guide surface 55 from the position where the guiding projection portion 550 has avoided the helical vertex portion 55A of the helical guide surface 55.

Incidentally, in the above-described embodiments, for example, as exemplified in FIGS. 4 and 6A-6D, one guide groove portion (also referred to as the slot) 56 is provided in the guide member 50. In each of the embodiments, without being limited thereto, two or more, that is, a plurality of the guide groove portions 56 may be provided in the guide member 50.

The connection device 200 according to the embodiment of the present disclosure is a connection device which connects an end portion of a first optical fiber cable FC1 as a first information transmission cable and an end portion of a second optical fiber cable FC2 as a second information transmission cable to each other. The connection device 200 can include the connection section 201 that holds the tubular connector fixing member 1405 holding the first optical fiber cable FC1 and having the projection portion 550 on the outer peripheral surface, and the tubular adapter fixing member 1603 that holds the second optical fiber cable FC2 and connects the end portion of the first optical fiber cable FC1 and the end portion of the second optical fiber cable FC2 to each other in accordance with insertion of the connector fixing member 1405. The adapter fixing member 1603 internally has the guide member 50. The guide member 50 is provided with the helical guide surface 55 formed in one direction in order to perform an operation in which the projection portion 550 of the connector fixing member 1405 is guided in a manner of gyrating around the central axis CL of the first optical fiber cable FC1 and the second optical fiber cable FC2 when the connector fixing member 1405 is inserted.

Accordingly, when the connector fixing member 1405 is inserted into the guide member 50 in order to connect the end portion of the first optical fiber cable FC1 and the end portion of the second optical fiber cable FC2 to each other, the projection portion 550 of the connector fixing member 1405 can be guided in a manner of gyrating along the helical guide surface 55 formed in one direction in the guide member 50 without confusing the gyratory direction.

In accordance with an exemplary embodiment, the projection portion 550 of the connector fixing member 1405 can be reliably guided in a manner of gyrating in only one direction along the helical guide surface 55 formed in one direction in the guide member 50. Accordingly, in the present disclosure, the connector fixing member 1405 and the guide member 50 can be smoothly coupled to each other, and the optical fiber cables can be reliably connected to each other while jamming is prevented from occurring in the connection device.

In contrast, in a case of a structure in the related art, a connector fixing member 405 gyrates in any direction of slope end surfaces 801A and 801B respectively in two directions. Since there are two slope surfaces, the angle of a sharp distal portion is significant, and there is concern that jamming may occur in the connection device.

As illustrated in FIGS. 13 and 14, the inner surface of the adapter fixing member 1603 is provided with the slope restraining projection portion 880 which restrains the connector fixing member 1405 from sloping with respect to the adapter fixing member 1603 when the connector fixing member 1405 is inserted into the guide member 50.

Accordingly, even though a force that tends to cause the connector fixing member 1405 to slope tends to be generated when the connector fixing member 1405 is intended to be inserted in the inner surface of the adapter fixing member 1603, the projection portion 880 causes a force that restrains sloping to act on the connector fixing member 1405. Therefore, the projection portion 880 restrains a force that tends to cause the connector fixing member 1405 to slope from being generated, and thus, a phenomenon can be further prevented in which the projection portion 550 of the connector fixing member 1405 is drawn into the inner surface of the guide member 50 and causes jamming therein.

As illustrated in FIGS. 15 and 16B, the outer peripheral surface of the connection section 201 is provided with the projection portion 411. The connection device 200 further can include the connection holding body 7 that internally accommodates the adapter fixing member 1603. The connection holding body 7 is provided with the helical guide groove portion 910, which guides the projection portion 411 in a manner of gyrating around the central axis CL. Before an operation in which the projection portion 550 of the connector fixing member 1405 is guided by the helical guide surface 55 in a gyrating manner starts, an operation in which the projection portion 411 of the connection section 201 is guided by the helical guide groove portion 910 in a gyrating manner is configured to start.

Accordingly, at the point of time the projection portion 411 of the connection section 201 enters the helical guide groove portion 910, the projection portion 550 of the connector fixing member 1405 has not yet bumped into the helical vertex portion of the helical guide surface 55 of the guide member 50, and a gyration guiding operation for the projection portion 411 of the connection section 201 is performed prior thereto along the helical guide groove portion 910 from the leading end portion of the helical guide groove portion 910 and proceeds. When the projection portion 550 hits the helical guide surface 55 of the guide member 50, the projection portion 550 is guided by the helical guide surface 55 in a gyrating manner. Accordingly, the projection portion 550 can be smoothly guided by the helical guide surface 55, and the end portions of the optical fiber cables can be reliably and smoothly connected to each other.

As illustrated in FIGS. 17, 18A and 18B, the distal portion on the outer peripheral surface of the connector fixing member 1405 is provided with the guide projection 890 which guides the connector fixing member 1405 in a manner of gyrating around the central axis CL by coming into contact with the helical guide surface 55 of the guide member 50 prior to the projection portion 550 when the connector fixing member 1405 is inserted into the guide member 50.

Accordingly, when the connector fixing member 1405 is inserted, the guide projection 890 hits the helical vertex portion of the helical guide surface 55 prior to the projection portion 550. Therefore, rotation along the helical guide surface 55 of the guide member 50 can be induced. In accordance with an exemplary embodiment, before the guiding projection portion 550 hits the helical vertex portion, the guide projection 890 bumps into the helical vertex portion. Therefore, the projection portion 550 can avoid hitting the helical vertex portion. Accordingly, the projection portion 550 can be caused to smoothly copy the helical guide surface 55 from the position where the projection portion 550 has avoided the helical vertex portion of the helical guide surface 55.

The imaging apparatus according to the present disclosure is the optical imaging apparatus 500, for example, provided with the above-described connection device. The imaging apparatus can include the catheter unit 100 that has the connection section 201; a drive device 1 that has the connection holding body 7, and emits light of a light source to a target through the first optical fiber cable FC1 and the second optical fiber cable FC2, and obtains return light from the target through the first optical fiber cable FC1 and the second optical fiber cable FC2; and the operation control apparatus 501 that forms an image of the target in accordance with a signal which is generated based on the return light and is sent from the drive device 1.

Accordingly, in the optical imaging apparatus 500 which is an example of the imaging apparatus provided with the connection device according to the embodiment of the present disclosure, when the connector fixing member 1405 is inserted into the guide member 50 in order to connect the end portion of the first optical fiber cable FC1 and the end portion of the second optical fiber cable FC2 to each other, the projection portion 550 of the connector fixing member 1405 can be guided in a manner of gyrating along the helical guide surface 55 formed in one direction in the guide member 50 without confusing the gyratory direction. That is, the projection portion 550 of the connector fixing member 1405 can be reliably guided in a manner of gyrating in only one direction along the helical guide surface 55 formed in one direction in the guide member 50. Accordingly, in the present disclosure, the connector fixing member 1405 and the guide member 50 can be smoothly coupled to each other, and the optical fiber cables can be reliably connected to each other while jamming is prevented from occurring in the connection device.

The connection device in each of the above-described embodiments and the imaging apparatus provided with the connection device are the optical coherence tomography apparatuses 500 (OCT) forming an image by using optical signals.

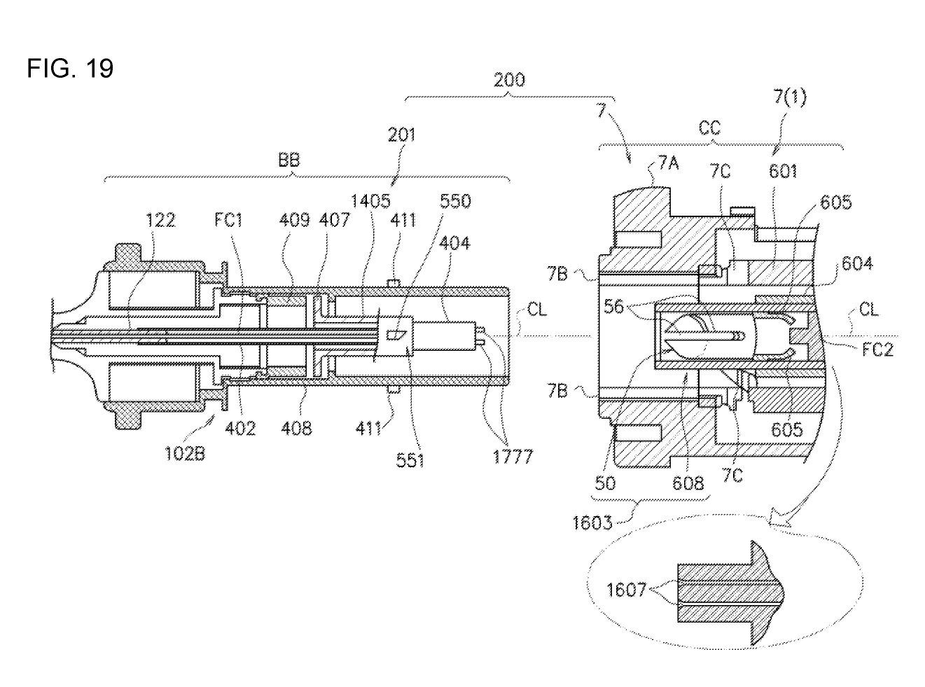

Here, as illustrated in FIG. 19, the connection device according to a fifth embodiment of the present disclosure is an intravascular ultrasound imaging system which forms an image by using ultrasound signals different from the optical coherence tomography apparatus 500 (OCT).

In the optical coherence tomography apparatus 500 illustrated in FIG. 3, the light transmitting and receiving unit 121 which transmits and receives measurement light is disposed inside the catheter sheath 101, and a lens is simply disposed at a position near the tube 103 ahead of the light transmitting and receiving unit 121. The light transmitting and receiving unit 121 only transmits light returned from a target object to the first optical fiber cable FC1.

In contrast, in the connection device according to the fifth embodiment of the present disclosure and the intravascular ultrasound imaging system serving as the imaging apparatus provided with the connection device, in place of the light transmitting and receiving unit 121, an acoustic wave transmitting and receiving unit 1121 formed of a piezoelectric material is disposed, and a transducer generating ultrasound waves is disposed in the acoustic wave transmitting and receiving unit 1121. An ultrasound signal which is transmitted and received by the acoustic wave transmitting and receiving unit 1121 is transmitted as an electrical signal through conductive cables by performing amplification of the signal, logarithmic conversion, and wave detection.

As illustrated in FIG. 19, an end portion 1777 of the conductive cable serving as the first information transmission cable and an end portion 1607 of the conductive cable serving as the second information transmission cable positioned on the central axis CL of the catheter connection holding body 7 can be connected to each other by being brought into contact with each other. In a case of the intravascular ultrasound imaging system, the cable has two or more terminals. Any set of the connection terminals of the cables may be connected to each other.

The fifth embodiment is not also limited to being provided with only one guide groove portion (referred to as the slot also) 56, and the plurality of guide groove portions 56 may be provided in the guide member 50.

The present disclosure is not limited to the above-described embodiments, and various types of changes can be made without departing from the scope of Claims. Each of the above-described embodiments according to present disclosure can be arbitrarily combined together. Each of the configurations in the above-described embodiments can be partially omitted or can be arbitrarily combined together so as to be different from that described above.

The detailed description above describes a connection device for connecting information transmission cables to each other, and an imaging apparatus provided with the connection device. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A connection device, which connects an end portion of a first information transmission cable and an end portion of a second information transmission cable to each other, the connection device comprising:
a connection section that holds a tubular connector fixing member holding the first information transmission cable and having a projection portion on an outer peripheral surface; and
a tubular adapter fixing member that holds the second information transmission cable and connects the end portion of the first information transmission cable and the end portion of the second information transmission cable to each other in accordance with insertion of the connector fixing member,
wherein the adapter fixing member internally has a guide member, and
wherein the guide member has a helical guide surface formed in one direction in order to perform an operation in which the projection portion of the connector fixing member is guided in a manner of gyrating around a central axis of the first information transmission cable and the second information transmission cable, when the connector fixing member is inserted.

2. The connection device according to claim 1,
wherein an inner surface of the adapter fixing member has a slope restraining projection portion, which restrains the connector fixing member from sloping with respect to the adapter fixing member when the connector fixing member is inserted into the guide member.

3. The connection device according to claim 1,
wherein an outer peripheral surface of the connection section has a projection portion; and
a connection holding body that internally accommodates the adapter fixing member,
wherein the connection holding body has a helical guide groove portion which guides the projection portion in a manner of gyrating around the central axis, and
wherein before an operation in which the projection portion of the connector fixing member is guided by the helical guide surface in a gyrating manner starts, an operation in which the projection portion of the connection section is guided by the helical guide groove portion in a gyrating manner.

4. The connection device according to claim 1,
wherein a distal portion on the outer peripheral surface of the connector fixing member has a guide projection which guides the connector fixing member in a manner of gyrating around the central axis by coming into contact with the helical guide surface of the guide member prior to the projection portion when the connector fixing member is inserted into the guide member.

5. The connection device according to claim 1,
wherein the first information transmission cable has an imaging core, and the imaging core generates an optical signal or an acoustic signal.

6. A connection device, which connects an end portion of a first information transmission cable and an end portion of a second information transmission cable to each other, the connection device comprising:
a tubular connector configured to hold the first information transmission cable and having a projection portion on an outer peripheral surface; and
a tubular adapter configured to hold the second information transmission cable and connects the end portion of the first information transmission cable and the end portion of the second information transmission cable to each other in accordance with insertion of the tubular connector, the tubular adapter internally having a guide member, and wherein the guide member has a helical guide surface formed in one direction in order to perform an operation in which the projection portion of the tubular connector is guided in a manner of gyrating around a central axis of the first information transmission cable and the second information transmission cable, when the tubular connector is inserted.

7. The connection device according to claim 6,
wherein an inner surface of the tubular adapter has a slope restraining projection portion, which restrains the tubular connector from sloping with respect to the tubular adapter when the tubular connector is inserted into the guide member.

8. The connection device according to claim 6,
wherein an outer peripheral surface of the tubular connector has a projection portion,
a connection holding body that internally accommodates the tubular adapter, wherein the connection holding body has a helical guide groove portion which guides the projection portion in a manner of gyrating around the central axis, and
wherein before an operation in which the projection portion of the tubular connector is guided by the helical guide surface in a gyrating manner starts, an operation in which the projection portion is guided by the helical guide groove portion in a gyrating manner.

9. The connection device according to claim 6,
wherein a distal portion on the outer peripheral surface of the tubular connector has a guide projection, which guides the tubular connector in a manner of gyrating around the central axis by coming into contact with the helical guide surface of the guide member prior to the projection portion when the tubular connector is inserted into the guide member.

10. The connection device according to claim 6,
wherein the first information transmission cable has an imaging core, and the imaging core generates an optical signal or an acoustic signal.

11. An imaging apparatus, the imaging apparatus comprising:
a connection device, which connects an end portion of a first information transmission cable and an end portion of a second information transmission cable to each other, the connection device including a connection section that holds a tubular connector fixing member holding the first information transmission cable and having a projection portion on an outer peripheral surface, and a tubular adapter fixing member that holds the second information transmission cable and connects the end portion of the first information transmission cable and the end portion of the second information transmission cable to each other in accordance with insertion of the connector fixing member, wherein the adapter fixing member internally has a guide member, and wherein the guide member has a helical guide surface formed in one direction in order to perform an operation in which the projection portion of the connector fixing member is guided in a manner of gyrating around a central axis of the first information transmission cable and the second information transmission cable, when the connector fixing member is inserted;
a catheter unit having the connection section;
a drive device having a connection holding body, the drive device configured to emit a signal to a target through the first information transmission cable and the second information transmission cable, and obtain a signal from the target through the first information transmission cable and the second information transmission cable; and
an operation control apparatus that forms an image of the target in accordance with a signal which is generated based on the signal from the target and is sent from the drive device.

12. The imaging apparatus according to claim 11,
wherein an inner surface of the adapter fixing member has a slope restraining projection portion, which restrains the connector fixing member from sloping with respect to the adapter fixing member when the connector fixing member is inserted into the guide member.

13. The imaging apparatus according to claim 11,
wherein an outer peripheral surface of the connection section has a projection portion; and
a connection holding body that internally accommodates the adapter fixing member, the connection holding body having a helical guide groove portion which guides the projection portion in a manner of gyrating around the central axis, and
wherein before an operation in which the projection portion of the connector fixing member is guided by the helical guide surface in a gyrating manner starts, an operation in which the projection portion of the connection section is guided by the helical guide groove portion in a gyrating manner.

14. The imaging apparatus according to claim 11,
wherein a distal portion on the outer peripheral surface of the connector fixing member has a guide projection which guides the connector fixing member in a manner of gyrating around the central axis by coming into contact with the helical guide surface of the guide member prior to the projection portion when the connector fixing member is inserted into the guide member.

15. The imaging apparatus according to claim 11, wherein the first information transmission cable has an imaging core, and the imaging core generates an optical signal or an acoustic signal.
